(19) United States
(12) Patent Application Publication
(10) Pub. No.: US 2010/0030027 A1
(43) Pub. Date: Feb. 4, 2010

(54) INTRAORAL MEDICAL DEVICE

(76) Inventors: Christophe Bastid, Marseille (FR); Patrick Bey, Cote D'Ivoire (FR); Christophe Poirot, Moyenmoutier (FR); Giorgio Bau, Le Mont (FR)

Correspondence Address: JEROME D. JACKSON (JACKSON PATENT LAW OFFICE), 211 N. UNION STREET, SUITE 100, ALEXANDRIA, VA, 22314, US

(21) Appl. No.: 12/373,446

(22) PCT Filed: Jul. 9, 2007
(86) PCT No.: PCT/FR2007/001165

§ 371 Date: Sep. 14, 2009

(30) Foreign Application Priority Data
Jul. 12, 2006 (FR) 0606360

(51) Int. Cl. A61B 1/24

(52) U.S. Cl. 600/120

(57) ABSTRACT

The present invention relates to an intraoral medical device comprising: • a mouth opener (2) formed by a tubular element (4) arranged to define an access to the oral cavity of the patient, • a tongue depressor (1) designed to be inserted removably into the tubular element (4) and having:—a channel (12) through which a breathable gas is injected and which opens out at the distal end of said tongue depressor,—a conduit through which surgical instruments are passed and which opens out at the distal end of said tongue depressor, the mouth opener (2) comprising an injection conduit (20) designed to be connected to a source of breathable gas and configured to inject said gas into the oral cavity of the patient when the tongue depressor (1) is not inserted in the tubular element (4) and to inject said gas into the injection channel (12) of said tongue depressor when the latter is inserted in said tubular element.

An object of the present invention is an intraoral medical device.

It relates to the general technical field of medical and surgical instruments employed during anesthesia or resuscitation of a patient, and more particularly those employed in upper tract endoscopic interventions.

During an endoscopy carried out via upper tract, it is often necessary to use an oxygen supply, which is usually effectuated by means of a tube inserted into the oral cavity or into the nostrils or in their immediate vicinity.

Intra-oral medical devices constituted by a mouth-opener fitted with a conduit adapted to inject oxygen into the oral cavity of the patient are known. Such devices are, for example, known from the documents U.S. Pat. No. 4,495,945 (KENNETH), GB 2,173,105 (BARNES) or WO 91/08012 (BORODY). However, these devices have the disadvantage that a substantial fraction of the supplied oxygen is lost, useless for the patient and dangerous for the operators (especially if an anesthetic gas is added) in the operating room.

Oxygen should therefore be ideally supplied as close as possible to the vocal cords. Thus, known are intra-oral medical devices constituted by a tongue-depressor comprising an oxygen injection channel opening at the oro-pharyngeal junction.

Such devices are described, for example, in the documents U.S. Pat. No. 2,127,215 (GWATHMEY), U.S. Pat. No. 2,705,959 (CAROL et al.), U.S. Pat. No. 3,756,244 (KINNEAR et al.), U.S. Pat. No. 4,881,542 (SCHMIDT et al.), GB 1,558,171 (BURCHELL), DE 201.03.524.U (GREBKOWSKI), WO 80/00538 (LUOMANEN), and WO 02/092144 (ELISHA MED. TECH. LTD).

However, the positioning of such devices is disturbing and often painful for patients. They are generally introduced once the patients are anesthetized and unconscious. But in this case, patients are not pre-oxygenated before the anesthesia, thereby increasing risks of hypoxemia.

Also known are intra-oral medical devices constituted by a mouth-opener adapted to receive a tongue-depressor comprising an oxygen injection channel opening at the oro-pharyngeal junction. Such devices are described, for example, in the documents U.S. Pat. No. 4,270,531 (BLACHLY et al.) or U.S. 2005/0217678 (McCORMICK et al.). However, such devices are not adapted for passage of surgical instruments and do not enable pre-oxygenation before anesthesia. In addition, the device described in U.S. 2005/0217678 (McCORMICK et al.) does not enable insertion of a tongue-depressor into the mouth-opener without disconnecting the oxygenation system from the latter.

The device closest to the invention is described in the document U.S. Pat. No. 4,848,331 (NORTHWAY-MEYER). This document discloses an intra-oral medical device comprising:

a mouth-opener formed by a tubular element arranged to define an access to the oral cavity of the patient,

a tongue-depressor adapted to be removabley inserted into the tubular element when the latter is positioned at the patient's mouth, the aforementioned tongue-depressor comprising

a injection channel, for a breathable gas, opening at the distal end of the aforementioned tongue-depressor,

a conduit, for the passage of surgical instruments, opening at the distal end of the aforementioned tongue-depressor.

Such a device enables pre-oxygenation of the patient at the beginning of the intervention, then insertion of the tongue-depressor into the tubular element after effective anesthesia to inject the oxygen at the oro-pharyngeal junction.

However, this device is not entirely satisfactory. In fact, during the surgical operations, it is necessary to connect the oxygen source onto a specific orifice positioned on the tongue-depressor. It is therefore necessary to have another specific orifice positioned on the respiratory mask during the pre-oxygenation phase. This device is in fact relatively complex and difficult to handle.

In view of all the disadvantages of the prior art, the principal technical problem envisioned to be solved by the invention is to enable, in a particularly simple manner, pre-oxygenation of the patient before anesthesia and then oxygenation of the patient by conveying oxygen to the oro-pharyngeal junction, without introducing additional equipment.

A goal of the invention is also to provide an intra-oral device adapted to aspirate oral or other secretions, without introducing additional equipment.

Another goal of the invention is to provide an intra-oral medical device of the type described in U.S. Pat. No. 4,848,331 (NORTHWAY-MEYER), less complex and able to be handled more easily.

The solution provided by the invention is an intra-oral medical device comprising:

a mouth-opener formed by a tubular element arranged to define an access to the oral cavity of the patient,

a tongue-depressor adapted to be removably inserted into the tubular element and comprising:

an injection channel, for a breathable gas, opening at the distal end of the aforementioned tongue-depressor,

a conduit, for the passage of surgical instruments, opening at the distal end of the aforementioned tongue-depressor,

and wherein the mouth-opener comprises an injection conduit adapted to be connected to a breathable gas source and configured to inject the aforementioned gas into the oral cavity of the patient when the tongue-depressor is not inserted into the tubular element and to inject the aforementioned gas into the injection channel of the aforementioned tongue-depressor when the latter is inserted in the aforementioned tubular element. It is thus possible to convey the breathable gas to the oro-pharyngeal junction without the need to connect the tongue-depressor to the gas source and without having to disconnect the latter from the mouth-opener.

According to an advantageous feature simplifying the design of the device object of the invention, the injection channel comprises an inlet orifice positioned opposing the outlet orifice of the injection conduit when the tongue-depressor is inserted into the tubular element.

According to another advantageous feature of the invention optimizing the flow of breathable gas, the interior wall of the injection channel located at the inlet orifice and the interior wall of the injection conduit located at the orifice output are beveled along a common direction.

According to yet another advantageous feature of the invention enabling efficient injection of the breathable gas during the pre-oxygenation phase, the injection conduit extends toward the rear of the tubular element so that its distal end, on which is arranged the outlet orifice, is positioned towards the back of the oral cavity when the mouth-opener is positioned.

According to another advantageous feature of the invention simplifying the design and enabling free access to the oral cavity of the patient, the injection conduit is integral with the tubular element and arranged laterally along one of the lower edges of the aforementioned tubular element.

According to another advantageous feature of the invention enabling limitation of gas losses during the pre-oxygenation phase, the outlet orifice of the injection conduit is configured so that the breathable gas is ejected towards the back of the oral cavity of the patient. And preferentially, the outlet orifice is positioned on the upper part of the injection conduit, the interior wall of the latter being inclined rearward at the aforementioned outlet orifice.

According to yet another advantageous feature designed to protect the comfort of the patient during the positioning of the device object of the invention, the distal end of the injection conduit is beveled.

According to another advantageous feature of the invention simplifying the design and enabling free passage of surgical instruments, the injection channel is integral with the tongue-depressor and positioned laterally along one of the interior edges of the conduit for the passage of surgical instruments.

According to yet another advantageous feature of the invention, the opener comprises a secretions aspiration conduit.

According to yet another advantageous feature of the invention simplifying the design and enabling free access to the oral cavity of the patient, the secretion aspiration conduit is integral with the tubular element and positioned laterally along one of the lower edges of the aforementioned tubular element.

According to yet another advantageous feature facilitating the handling of the device object of the invention, the aspiration conduit comprises an inlet orifice located on the external face of the mouth-opener and on which is arranged a nozzle designed to be connected an aspiration device.

According to yet another advantageous feature of the invention enabling efficient aspiration of the secretions, the secretions aspiration conduit extends toward the rear of the tubular element so that its distal end, on which is arranged an aspiration orifice, is positioned towards the back of the oral cavity when the mouth-opener is positioned at the mouth of the aforementioned patient.

According to yet another advantageous feature of the invention designed to ensure efficient aspiration of the secretions, the distal end of the aspiration conduit is beveled, aspiration orifice being arranged on the beveled face of the aforementioned distal end so that it is directed downwards.

According to yet another advantageous feature of the invention enabling aspiration of the secretions accumulated at the entrance to the oral cavity, the aspiration conduit comprises an aspiration orifice arranged on a wall of the tubular element. And to avoid this orifice being blocked during the positioning of the tongue depressor, the latter comprises an opening positioned opposing the aspiration orifice when the aforementioned tongue-depressor is inserted into the tubular element.

According to yet another advantageous feature of the invention, comprises a secretions aspiration orifice.

According to yet another advantageous feature of the invention enabling aspiration of the secretions at the tongue-depressor without having to connect the latter to an aspiration device, the inlet orifice of the aspiration conduit of the tongue-depressor is positioned opposing an opening arranged on the aspiration conduit of the mouth-opener when the aforementioned tongue-depressor is inserted into the tubular element.

According to another feature of the invention to facilitate the withdrawal of the tongue-depressor, the latter comprises two fins 11 positioned on both sides of its external face, the aforementioned fins being configured to be mutually arranged at the openings on both sides of the external face of the mouth-opener.

Other advantages and features of the invention will better appear upon reading the description of a preferred implementation mode that will follow, in reference to the attached drawings, made by way of guiding non-limiting examples and in which:

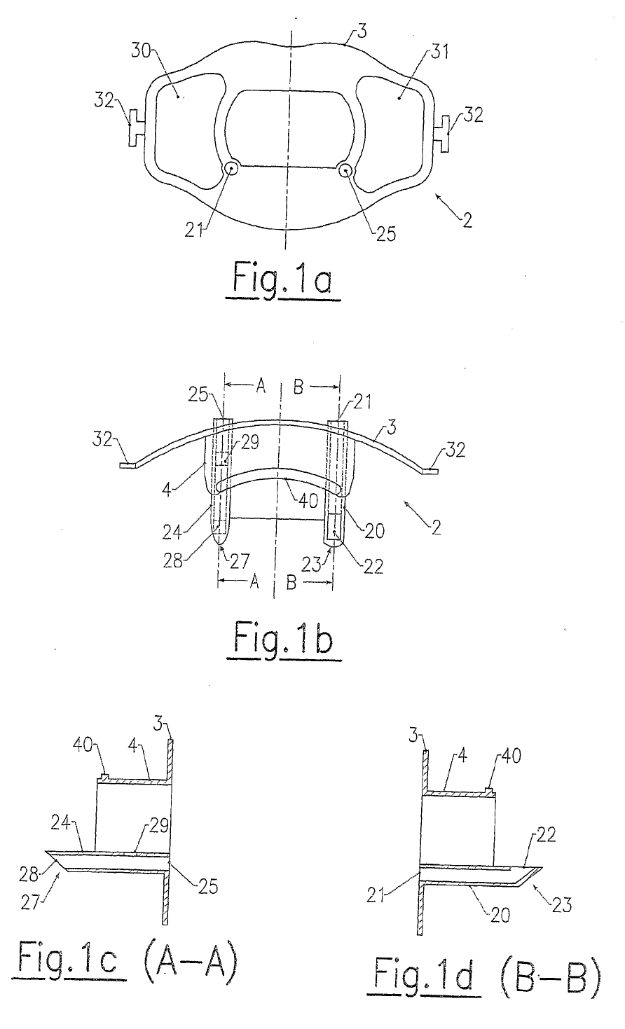

FIG. 1a is a schematic front view of the mouth-opener,

FIG. 1b is a schematic top view of the mouth-opener of FIG. 1a,

FIG. 1c is a cross-section view along A-A of the mouth-opener of FIG. 1b showing schematically the arrangement of the secretion aspiration conduit,

FIG. 1d is a cross-section view along B-B of the mouth-opener of FIG. 1b showing schematically the arrangement of the breathable gas injection conduit,

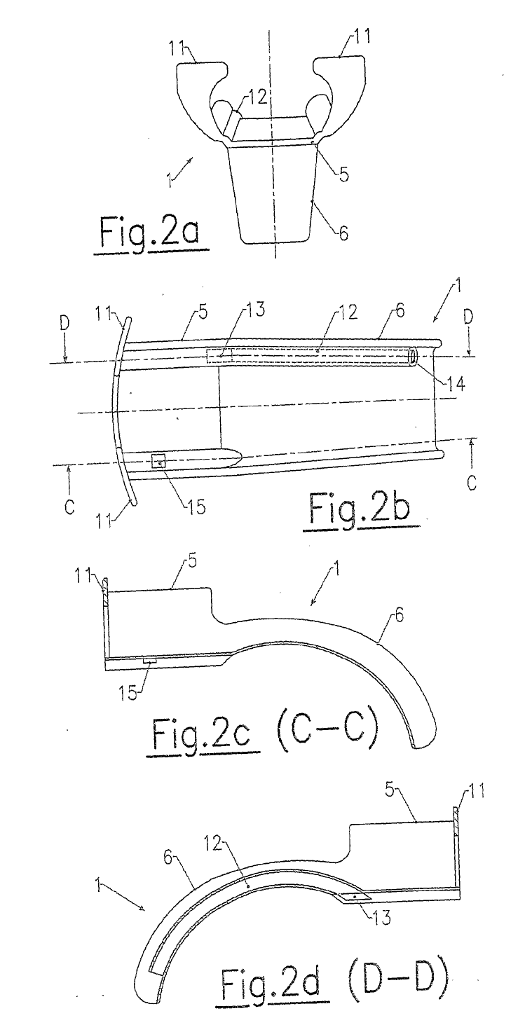

FIG. 2a is a schematic front view of the tongue-depressor,

FIG. 2b is a schematic top view of the tongue-depressor of FIG. 2a,

FIG. 2c is a cross-section view along C-C of the tongue-depressor of FIG. 2b,

FIG. 2d is a cross-section view along D-D of the tongue-depressor of FIG. 2b showing schematically the arrangement of the breathable gas injection channel,

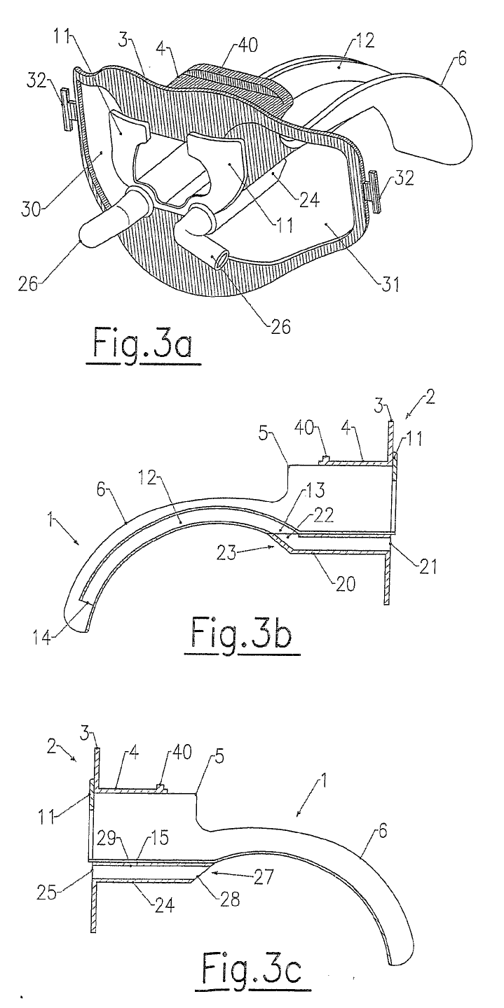

FIG. 3a is a schematic perspective view of the intra-oral device in accordance with the invention, the tongue-depressor being inserted into the mouth-opener,

FIG. 3b is a vertical cross-section view of the intra-oral device of FIG. 3a showing the connection between the injection conduit of the mouth-opener and the injection channel of the tongue-depressor,

FIG. 3c is a vertical cross-section view of the intra-oral device of FIG. 3a showing the connection between the secretions aspiration conduit of the mouth-opener and the tongue-depressor.

The device object of the invention is constituted by a single use tongue-depressor 1 designed to be mounted on a medical mouth-opener 2 for upper tract endoscopy.

The mouth-opener 2 is first positioned at the patient's mouth when the latter is conscious. Once the patient is anesthetized and unconscious, the tongue-depressor 1 is inserted into the mouth-opener 2.

The mouth-opener 2 is made of a material suitable for medical use. In practice, it is obtained by molding of thermoplastic polyethylene-based polymer. Other materials and similar manufacturing processes may be employed.

Referring to FIGS. 1a, 1b, 1c and 1d, the mouth-opener 2 comprises a tubular element 4 inserted between the teeth of the patient so as to prevent the mouth from closing.

In an implementation variation not shown, the mouth-opener 2 can also be of the type described in U.S. Pat. No. 4,848,331 (NORTHWAY-MEYERS).

The tubular element 4 is arranged to define an access to the oral cavity of the patient. The physician or surgeon can manipulate surgical instruments through the aforementioned tubular element.

In practice, the tubular element 4 has a general rectangular shape, slightly curved at the distal part in order to adapt to the morphology of the mouth. The dimensions of the tubular element 4 can vary depending on the size of the mouth of the patient so that its positioning is as comfortable as possible.

The tubular element 4 advantageously comprises means for maintaining the mouth-opener 2 in position in the mouth of the patient during the procedure.

Referring to the preferred implementation mode shown in FIGS. 1b, 1c and 1d, the tubular element 4 comprises a protruding part 40 designed to be placed behind the teeth so that the mouth-opener 2 can no longer escape from the mouth of the patient once positioned.

Grooves can also be provided in which the teeth are wedged or a relatively flexible cover can be employed in which the teeth are embedded.

The mouth-opener 2 advantageously comprises an external part 3 pressed against the face of the patient.

Referring to the attached figures, this external part 3 is arranged at the proximal portion of the tubular element 2. Two openings 30 and 31 are preferably arranged on both sides of the external face of the mouth-opener 2.

On each side of the external part 3 are positioned <<T>> shaped elements 32 (FIG. 1a) designed to receive an elastic band extending around the head of the patient so as to maintain the mouth-opener 2 in position.

Referring to FIGS. 1a, 1b, 1c and 1d, mouth-opener 2 comprises an injection conduit 20 adapted to be connected to a source of breathable gas and configured to inject the aforementioned gas into the oral cavity of the patient when the tongue-depressor 1 is not inserted into the tubular element 4. The presence of such an injection conduit enables pre-oxygenation of the patient in a simple and particularly efficient manner before the patient is anesthetized.

According to the preferred implementation mode shown in the attached figures, the injection conduit 20 is integral with the tubular element 4 during molding and arranged laterally along one of its lower edges so as to allow free access to the oral cavity and clear the way for surgical instruments. However, it can be implemented using any technique to achieve an equivalent result, for example by using a separate tube mounted after molding of the mouth-opener.

The injection conduit 20 comprises an inlet orifice 21 located on the external face of the mouth-opener 2 and on which can be arranged a nozzle 26 (FIG. 3a), removable or not, enabling easy connection to a source of oxygen or any other breathable gas.

Referring more particularly to FIGS. 1b and 1d, the injection conduit 20 extends toward the rear of the tubular element 4 so that its distal end 23, on which is arranged the outlet orifice 22, is positioned towards the back of the oral cavity when the mouth-opener is positioned in the mouth of the patient. The breathable gas is thus injected relatively far from the entrance of the oral cavity of the patient to avoid wasteful losses.

In accordance with FIG. 1d, the distal end 23 of the injection conduit 20 is advantageously beveled so as not to disturb or injure the patient when the mouth-opener 2 is introduced into the oral cavity.

The outlet orifice 22 is arranged on the distal end 23 and configured so that the breathable gas is ejected towards the back of the oral cavity to avoid any wasteful loss. In practice, the outlet orifice 22 is positioned on the upper part of the injection conduit 20 and the interior wall of the latter is inclined rearward at the aforementioned outlet orifice.

Referring to FIGS. 1a, 1b, 1c and 1d, the mouth-opener 2 preferably comprises a secretion aspiration conduit 24 designed to evacuate the different secretions (saliva, blood, . . . ) present in the oral cavity of the patient and liable to disrupt the surgical workflow.

According to a preferred implementation mode, the aspiration conduit 24 is integrated at the time of the molding the tubular element 4. It is laterally arranged along a lower edge of the tubular element 4, advantageously opposing the injection conduit 20, in order to allow free access to the oral cavity and clear the way for surgical instruments. However, it can be implemented by using any technique enabling achievement of an equivalent result, for example by using a separate tube mounted after the molding of the mouth-opener 2.

The aspiration conduit 24 comprises an inlet orifice 25 located on the external face of mouth-opener 2 and on which can be arranged a nozzle 26 (FIG. 3a), removable or not, enabling easy connection of it to a pump-type aspiration device.

Referring more particularly to FIGS. 1b and 1c, the aspiration conduit 24 extends toward the back of the tubular element 4 so that the distal end 27, on which is arranged the aspiration orifice 28, is positioned towards the back of the oral cavity when the mouth-opener 2 is positioned. This configuration ensures efficient aspiration of secretions.

In accordance with FIG. 1c, the distal end 27 of the aspiration conduit 24 is advantageously beveled so as not to disturb or injure the patient when the mouth-opener 2 is positioned. Advantageously, the aspiration orifice 28 is arranged on the beveled face of the distal end 27 so that it is directed downwards to make the aspiration more efficient.

Referring to FIGS. 1a, 1b and 1c, the aspiration conduit 24 comprises another aspiration orifice 29 arranged on a wall of the tubular element 4. This orifice is designed to aspirate the secretions accumulated at the entrance of the oral cavity and cannot be aspirated through the orifice 28.

The tongue-depressor 1 is made of a material suitable for medical use. It is advantageously made by molding of thermoplastic polyethylene-based polymer. Other materials and similar manufacturing processes may be employed.

Referring to FIGS. 2a, 2b, 2c and 2d, the tongue-depressor 1 is formed:

from a base 5 configured to be removably interlocked in the tubular element 4 of the mouth-opener 2,

and from a canula 6 adapted to penetrate into the mouth to the oro-pharyngeal junction.

In practice, the base 5 has a shape complementary to the tubular element 4. The canula 6 has a curved shape adapted to serve as a tongue-holder and prevent tipping of the posterior of the tongue. It thus results in the forcing of the tongue down and forward in order to ensure freedom of the upper airways.

The withdrawal of the tongue-depressor 1 is facilitated by two extraction fins 11 positioned on both sides of the external face of the aforementioned tongue-depressor. To facilitate the extraction of the tongue-depressor 1 and the prehension of the two fins 11, the two fins 11 are configured to be mutually arranged at the openings 30 and 31 of the external part 3 of the mouth-opener 2 (FIG. 3a).

The cross-section of the tongue-depressor 1 is preferably “U” shaped, open at the top along its entire length so as to form a channel from the exterior of the oral cavity toward the oro-pharyngeal junction and allowing the passage of endoscopes or any other surgical instruments, while ensuring their guidance. In an implementation variation not shown, tongue-depressor 1 has a tubular cross-section along its entire length.

Referring to FIGS. 2a, 2b and 2d, the tongue-depressor 1 comprises an injection channel 12 for injecting a breathable gas opening at the distal end of the aforementioned tongue-depressor.

The injection channel 12 comprises an outlet orifice 14 positioned in the oro-pharyngeal junction when the tongue-depressor 1 is inserted in the tubular element 4 and the latter is positioned in the mouth of the patient.

According to a preferred implementation mode, the injection channel 12 is integral with the tongue-depressor 1 at the time of molding in the conduit for the passage of surgical instruments. It is advantageously molded laterally along one of the interior edges of this conduit to allow free access to the oral cavity and clear the way for instruments to be introduced through the mouth of the patient. However, it can be implemented using any technique enabling attainment of an equivalent result, for example by using a separate tube mounted after the molding of the tongue-depressor 1.

In accordance with FIGS. 2d and 3b, the injection channel 12 comprises an inlet orifice 13 positioned opposing the outlet orifice of the injection conduit 20 when it is inserted into the tubular element 4. Thus, it is not necessary to disconnect the connector 26 of the mouth-opener 2 to connect to the inlet orifice 13 of the injection channel 12. In fact, the act of inserting the tongue-depressor 1 into the tubular element 4 results in directing the breathable gas into the injection channel 12. Other provisions enabling supplying of the injection channel 12 in an equivalent manner could of course be employed.

In order to simplify the design of the device object of the invention, the outlet orifice 13 is arranged at the base 5.

To avoid a sudden change of direction of the flow of breathable gas when entering the injection channel 12, the interior wall of the aforementioned channel located at the inlet orifice 13 and the interior wall of the injection conduit 20 located at the outlet 22 are beveled along a common direction (FIG. 3b).

Referring to the attached figures, the injection conduit 20 comprises a protruding part in the internal part of the tubular element 4 and the tongue-depressor 1 comprises a hollow part adjusted to the shape of the aforementioned protruding part. The outlet orifice 22 of the injection conduit 20 is advantageously arranged on the protruding part of the aforementioned injection conduit and the input orifice 13 of the injection channel 12 is advantageously arranged on the hollow part of the tongue-depressor 1.

The protruding part and the hollow part have complementary shape and are adjusted to ensure a good seal at the connection between the inlet orifice 13 and the outlet orifice 22. A joint type means of sealing may be provided at the outlet orifice 22 and/or the inlet orifice 13.

Having a tongue-depressor 1 with a hollow part adapted to translate on a protruding part positioned in the tubular element 4 also enables assurance of efficient guidance of the aforementioned tongue-depressor during its positioning.

With reference to FIGS. 2a, 2b and 2c, the tongue-depressor 1 comprises an opening 15 positioned opposing the aspiration orifice 29 arranged in the interior of the tubular element 4 when it is inserted in the aforementioned tubular element (figure 3c). The opening 15 is positioned in the base 5 of the tongue-depressor 1. Thus, the positioning of the tongue-depressor 1 in the tubular element 1 does not result in plugging the aspiration orifice 29. In all cases, the arrangement of the aspiration orifice 28 on the beveled side of the distal end 27 of the aspiration conduit 24 means that this orifice 28 is not likely to be blocked by the insertion of the tongue-depressor 1.

Referring to the attached figures, the secretions aspiration conduit 24 comprises a protruding part in the internal part of the tubular element 4 and the tongue-depressor 1 comprises a hollow part adjusted to the shape of the protruding part. The aspiration orifice 29 is arranged on the protruding part of the aspiration conduit 24 and the opening 15 is arranged on the hollow part of the tongue-depressor 1.

The protruding part and the hollow part have complementary shape and are adjusted to ensure a good seal at the connection between the aspiration orifice 29 and the opening 15. A joint type means of sealing can possibly be provided at the aspiration orifice 29 and/or the opening 15.

In an implementation variation not shown, the tongue-depressor 1 is equipped with a secretions aspiration conduit integrated during molding into the conduit for passage of surgical instruments, opposing the injection channel 12. This aspiration conduit advantageously comprises an aspiration orifice arranged to aspirate the secretions accumulated deeper in the oral cavity.

In the same manner as described above, the inlet orifice of the aspiration conduit of the tongue-depressor 1 is advantageously positioned opposing the opening 15 when the aforementioned tongue-depressor is inserted into the tubular element 4. Thus, it is not necessary to disconnect the connector 26 of the mouth-opener 2 to connect to the inlet orifice of the aspiration conduit of the tongue-depressor 1.

1. An intraoral medical device comprising:
a mouth-opener formed by a tubular element arranged to define an access to the oral cavity of the patient,
a tongue-depressor adapted to be removably inserted into the tubular element and comprising:
an injection channel, for a breathable gas, opening at the distal end of the aforementioned tongue-depressor,
a conduit, for the passage of surgical instruments, opening at the distal end of the aforementioned tongue-depressor,
characterized by the fact that mouth-opener comprises an injection conduit adapted to be connected to a breathable gas source and configured to inject the aforementioned gas into the oral cavity of the patient when the tongue-depressor is not inserted into the tubular element and to inject the aforementioned gas into the injection channel of the aforementioned tongue-depressor when the latter is inserted in the aforementioned tubular element.

2. A device according to claim 1, wherein the injection channel comprises an inlet orifice positioned opposing the outlet orifice of the injection conduit when the tongue-depressor is inserted into the tubular element.

3. A device according to claim 2, wherein the interior wall of the injection channel located at the inlet orifice and the interior wall of the injection conduit located at the outlet are beveled along a common direction.

4. A device according to claim 1, wherein the injection conduit is integral with the tubular element and positioned laterally along one of the lower edges of the aforementioned tubular element.

5. A device according to claim 1, wherein the injection conduit extends toward the rear of the tubular element so that the distal end, on which is arranged the outlet orifice, is positioned towards the back of the oral cavity when the mouth-opener is positioned in the mouth of the patient.

6. A device according to claim 1, wherein the outlet orifice of the injection conduit is configured so that the breathable gas is ejected toward the back of the oral cavity when the mouth-opener is positioned at the mouth of the patient.

7. A device according to claim 6, wherein the outlet orifice is positioned on the upper part of the injection conduit, the interior wall of the latter being inclined rearward at the aforementioned outlet orifice.

8. A device according to claim 1, wherein the distal end of the injection conduit is beveled.

9. A device according to claim 1, wherein the injection channel is integral with the tongue-depressor and positioned laterally along one of the interior edges of the conduit for passage of surgical instruments.

10. A device according to claim 1, wherein the mouth-opener comprises a secretions aspirations conduit.

11. A device according to claim 10, wherein the aspiration conduit is integral with the tubular element and positioned laterally along one of the lower edges of the aforementioned tubular element.

12. A device of claim 10, wherein the aspiration conduit comprises an inlet orifice located on the external face of the mouth-opener and on which is arranged a nozzle designed to be connected to an aspiration device.

13. A device according to claim 10, wherein the aspiration conduit extends toward the rear of the tubular element so that its distal end, on which is arranged an aspiration orifice, is positioned towards the back of the oral cavity when the mouth-opener is positioned at the mouth of the patient.

14. A device according to claim 13, wherein the distal end of the aspiration conduit is beveled, the inlet orifice being arranged on the beveled face of the aforementioned distal end so that it is directed downwards.

15. A device according to claim 10, wherein the aspiration conduit comprises an aspiration orifice arranged on a wall of the tubular element.

16. A device according to claim 15, wherein the tongue-depressor comprises an opening positioned opposing the aspiration orifice when the aforementioned tongue-depressor is inserted into the tubular element.

17. A device according to claim 1, wherein the tongue-depressor is equipped with a secretions aspiration conduit.

18. A device according to claim 17, wherein the inlet orifice of the aspiration conduit of the tongue-depressor is positioned opposing an opening arranged on the aspirations conduit of the mouth-opener when the aforementioned tongue-depressor is inserted into the tubular element.

19. A device according to claim 1, wherein the tongue-depressor comprises two fins positioned on both sides of its external surface, the aforementioned fins being configured to be mutually arranged at the two openings positioned on both sides of the external face of the mouth-opener.
